(19) United States
(12) Patent Application Publication
(10) Pub. No.: US 2023/0335227 A1
(43) Pub. Date: Oct. 19, 2023

(54) Molecule Designing Method, Device and Program

(71) Applicant: Asahi Kasei Kabushiki Kaisha, Tokyo (JP)

(72) Inventors: Hiroyuki Tsujimoto, Tokyo (JP); Natsumi Yamakawa, Tokyo (JP); Ippei Kimura, Tokyo (JP); Hideki Yamamoto, Osaka (JP)

(73) Assignee: Asahi Kasei Kabushiki Kaisha, Tokyo (JP)

(21) Appl. No.: 18/123,370

(22) Filed: Mar. 20, 2023

(30) Foreign Application Priority Data
Apr. 14, 2022 (JP) 2022 067155
Dec. 27, 2022 (JP) 2022 209321

(51) Int. Cl. G16C 20/50

(52) U.S. Cl. CPC G16C 20/50

(57) ABSTRACT

It is intended to provide a method, a device, and a program that are capable of outputting a molecule designing result having more appropriate performance in the range of molecule designing with which synthesis is realistically possible. A method executes, by a device, a sensitivity estimation step of inputting candidate information A, information B, and reference information C to a model to output sensitivity information D of a receptor constituted by using a polymer for an analyte, the candidate information A being related to the polymer, the information B being related to the analyte, the reference information C being related to a film constitution of the receptor.

### BACKGROUND OF THE INVENTION

### Field of the Invention

The present invention relates to a method, a device, and a program of molecule designing.

### Description of the Related Art

To obtain a novel material that satisfies a certain function, technical experts perform molecule designing of the novel material through trial and error based on tacit knowledge such as experience and ideas. For example, to design a molecule having a certain function, technical experts roughly predict a molecular skeleton based on tacit knowledge and technological common knowledge, actually synthesize a group of compounds having the molecular skeleton and having variation in substituents and the like, and check whether the group of compounds has the desired function by validation experiment.

In such a conventional method, whether a molecule to be actually designed has a desired function is yet to be confirmed at the stage of molecular skeleton prediction, and validation experiment of a synthesized compound is needed. Since a significant amount of time is normally needed for compound synthesis and function validation experiment, a large amount of time is needed for trial and error in conventional novel material molecule designing. Thus, molecule designing by using information processing technologies has been attempted, and for example, Belmares et al., Vol. 25, No. 15, pp 1814-1826, Journal of Computational Chemistry discusses evaluation of the response of a polymer sensor by using a Hildebrandt solubility parameter (SP value) and a Hansen solubility parameter (HSP value).

Recently, a membrane surface stress sensor (MSS) has been attracting attention as an olfactory sensor. The membrane surface stress sensor has a structure in which a receptor is provided on a MEMS sensor array, and when the receptor expands due to adsorption and internal diffusion of analyte in a receptor, stress that the expansion provides to a sensing unit of the sensor is electrically detected as an electric resistance or the like.

### SUMMARY OF THE INVENTION

To detect various odors with such a membrane surface stress sensor, a receptor having high adsorption sensitivity and a high expansion rate needs to be designed for each analyte (odor molecule) to be detected. However, it is difficult to design a receptor having appropriate detection performance for each odor molecule, of which several hundred thousand kinds are estimated to exist. In the field of material development, in particular, technical experts repeat trial and error as in the above-described conventional method to design a molecule that has a certain function from which one receptor is produced. Thus, new receptor designing technologies and production technologies are needed in order to propagate a membrane surface stress sensor as an olfactory sensor.

For such a technological problem, for example, if it were possible to quantify tacit knowledge and technological common knowledge of technical experts and perform molecule designing at an appropriate accuracy based on a function required for a novel material, we could expect acceleration of material development and significant reduction of development costs.

However, although molecule designing by using information processing technologies has been attempted as disclosed in Belmares et al., Vol. 25, No. 15, pp 1814-1826, Journal of Computational Chemistry, actual molecule synthesis and result verification have not been achieved and the accuracy of molecule designing cannot be evaluated in many cases. Furthermore, a molecule structure obtained by using information processing technologies is not based on consideration of a synthesis path, and thus when a molecule is to be actually synthesized to verify its function, it is extremely difficult to synthesize an output molecular skeleton and the verification is often impossible. Thus, the conventional molecule designing method using information processing technologies can be hardly used in the real world.

Therefore, in the field of material development, in order to replace the conventional molecule designing method with information processing technologies or provide information to be consulted by a developer with information processing technologies, it is desired to obtain a system that is capable of outputting a molecule designing result having more appropriate performance in the range of molecule designing with which synthesis is realistically possible.

The present invention is made in view of the above-described problem point and is intended to provide a method, a device, and a program that are capable of outputting a molecule designing result having more appropriate performance in the range of molecule designing with which synthesis is realistically possible.

Specifically, the present invention is as follows.

[1] A method that executes, by a device, a sensitivity estimation step of inputting candidate information A, information B, and reference information C to a model to output sensitivity information D of a receptor constituted by using a polymer for an analyte, the candidate information A being related to the polymer, the information B being related to the analyte, the reference information C being related to a film constitution of the receptor.
[2] The method according to [1], in which the information B comprises a one-dimensional solubility parameter H2-1 of the analyte.
[3] The method according to [1], in which the information B comprises a three-dimensional solubility parameter H2-2 of a London dispersion force term, a dipole term, and a hydrogen bond term of the analyte.
[4] The method according to [1], in which the information B comprises a Hansen solubility parameter H2-3 of the analyte.
[5] The method according to any one of [1] to [4], in which

in the sensitivity estimation step, information F related to mutual interaction between the polymer and the analyte is additionally input to the model, and

the information F comprises a x parameter.

[6] The method according to any one of [1] to [4], in which

in the sensitivity estimation step, information F related to mutual interaction between the polymer and the analyte is additionally input to the model, and

the information F comprises a x parameter and/or a Ra parameter.

[7] The method according to any one of [1] to [6], in which in the sensitivity estimation step, the model additionally outputs recommendation information C′ related to the film constitution of the receptor.
[8] The method according to any one of [1] to [7], further executing, before the sensitivity estimation step:
a step of producing a candidate for a molecule structure of the polymer; and
a step of calculating a solubility parameter H1 of the polymer based on the molecule structure produced,

in which the candidate information A comprises the solubility parameter H1 calculated.

[9] The method according to [8], in which the molecule structure comprises a functional group included in the polymer and/or a copolymerization ratio of the polymer.
[10] The method according to [9], further executing:

a step of outputting the sensitivity information D for each of a plurality of pieces of the candidate information A;

a step of specifying the sensitivity information D that satisfies a predetermined condition among the plurality of pieces of output sensitivity information D; and

a molecule structure estimation step of outputting, as information E related to the molecule structure of the polymer constituting the receptor, the molecule structure of the polymer of the candidate information A corresponding to the specified sensitivity information D.

[11] The method according to any one of [1] to [8], further executing, before the sensitivity estimation step, a step of producing a candidate for a solubility parameter H1 of the polymer based on the information B without specifying the molecule structure of the polymer, in which the candidate information A comprises the solubility parameter H1 produced.
[12] The method according to [11], further executing:

a step of outputting the sensitivity information D for each of a plurality of pieces of the candidate information A;

a step of specifying the sensitivity information D that satisfies a predetermined condition among the plurality of pieces of output sensitivity information D;

a step of estimating the molecule structure of the polymer based on the solubility parameter H1 of the polymer of the candidate information A corresponding to the specified sensitivity information D; and

a molecule structure estimation step of outputting the estimated molecule structure of the polymer as information E related to the molecule structure of the polymer constituting the receptor.

[13] A device comprising an estimation unit configured to input candidate information A, information B, and reference information C to a model to output sensitivity information D of a receptor constituted by using a polymer for an analyte, the candidate information A being related to the polymer, the information B being related to the analyte, the reference information C being related to a film constitution of the receptor.
[14] The device according to [13], in which

the estimation unit additionally inputs, to the model, information F related to mutual interaction between the polymer and the analyte, and

the information F comprises a x parameter and/or a Ra parameter.

[15] A program configured to cause a device to execute a sensitivity estimation step of inputting candidate information A, information B, and reference information C to a model to output sensitivity information D of a receptor constituted by using a polymer for an analyte, the candidate information A being related to the polymer, the information B being related to the analyte, the reference information C being related to a film constitution of the receptor.
[16] The program according to [15], in which

in the sensitivity estimation step, information F related to mutual interaction between the polymer and the analyte is additionally input to the model, and

the information F comprises a x parameter and/or a Ra parameter.

According to the present invention, it is possible to provide a method, a device, and a program that are capable of outputting a molecule designing result having more appropriate performance in the range of molecule designing with which synthesis is realistically possible.

### BRIEF DESCRIPTION OF THE DRAWINGS

FIG. 1 is an example of a block diagram illustrating a functional configuration of a device according to the present embodiment;

FIG. 2A is a schematic view illustrating an example of information A according to the present embodiment;

FIG. 2B is a schematic view illustrating an example of information B according to the present embodiment;

FIG. 2C is a schematic view illustrating an example of information C according to the present embodiment;

FIG. 2D is a schematic view illustrating a list of correspondence between a known monomer unit and the solubility parameter H1 of the monomer unit;

FIG. 3A is a schematic view illustrating an example of a flowchart of a processing method according to the present embodiment;

FIG. 3B is a schematic diagram illustrating an example of a flowchart illustrating specific processing at Step S303 in FIG. 3A;

FIG. 3C is a schematic diagram illustrating an example of a flowchart illustrating specific processing at Step S303 in FIG. 3A;

FIG. 3D is a schematic diagram illustrating an example of a flowchart illustrating specific processing at Step S308 in FIG. 3A;

FIG. 4 is a schematic view illustrating an aspect of a case in which information E is output to another device;

FIG. 5A is a schematic view illustrating an example of the flowchart of the processing method according to the present embodiment;

FIG. 5B is a schematic diagram illustrating an example of a flowchart illustrating specific processing at Step S503 in FIG. 5A;

FIG. 5C is a schematic diagram illustrating an example of a flowchart illustrating specific processing at Step S503 in FIG. 5A;

FIG. 5D is a schematic diagram illustrating an example of a flowchart illustrating specific processing at Step S507 in FIG. 5A;

FIG. 5E is a schematic diagram illustrating an example of a flowchart illustrating specific processing at Step S509 in FIG. 5A;

FIG. 5F is a schematic diagram illustrating an example of a flowchart illustrating specific processing at Step S509 in FIG. 5A;

FIG. 6A is a diagram illustrating a result of an example; and

FIG. 6B is a diagram illustrating a result of a comparative example.

### DETAILED DESCRIPTION OF THE PREFERRED EMBODIMENTS

An embodiment of the present invention (hereinafter referred to as “the present embodiment”) will be described below in detail, but the present invention is not limited thereto and may be modified in various manners without departing from the scope thereof.

1. Sensor

First, an example of a membrane surface stress sensor for which a receptor is used will be described below. The membrane surface stress sensor is provided with a receptor on a sensor body. With the sensor body, the sensor detects a change that occurs to a physical parameter when the receptor adsorbs and diffuses an analyte (sample molecule) into the film. The kind of sensor body usable in the sensor of the present embodiment is not particularly limited but may be selected as appropriate in accordance with a physical parameter kind.

Such a physical parameter is not particularly limited but examples thereof include surface stress, stress, force, surface tension, pressure, mass, elasticity, Young's modulus, Poisson's ratio, resonance frequency, frequency, volume, thickness, viscosity, density, magnetic force, magnetic charge, magnetic field, magnetic flux, magnetic flux density, electric resistance, quantity of electricity, dielectric constant, electric power, electric field, electric charge, current, voltage, potential, mobility, electrostatic energy, capacitance, inductance, reactance, susceptance, admittance, impedance, conductance, plasmon, refractive index, luminous intensity, temperature, and other various physical parameters.

An applicable sensor body of another kind is, for example, a quartz crystal microbalance device (QCM device). The QCM device is a mass sensor configured to measure a small amount of mass change by utilizing a characteristic that, when a material is adsorbed on the surface of a quartz crystal electrode to which an alternating-current electric field is applied, resonance frequency decreases in accordance with the mass, viscoelasticity, or the like of the adsorbed material, and the QCM device can perform in-situ measurement. When the sensor of the present embodiment is applied as such a QCM device, for example, a receptor is formed on the surface of the electrode so that a mass change that occurs when the receptor adsorbs an analyte is detected by the sensor body and a signal is output. Various conductive materials are applicable as the QCM electrode, and a receptor in the present embodiment can be used as the QCM electrode when the receptor has conductivity.

When used in the present specification, the meaning of the term “adsorption” includes a phenomenon that the concentration of another material (to be adsorbed) at the interface of an object becomes higher than in surroundings and includes not only physical adsorption but also chemical adsorption due to a chemical bond and a biochemical effect.

1.1. Receptor

The receptor is not particularly limited and contains, for example, a polymer constituted by a certain monomer unit, and may contain a filler as necessary. When the receptor contains a filler, it is possible to adjust a parameter related to physical strength such as the Young's modulus of the receptor. Accordingly, it is possible to control the volume expansion rate of the sensor, thereby allowing sensitivity to be increased, for example.

The polymer constituting the receptor causes volume expansion or the like through mutual interaction with an analyte, and the filler, a void space, and the like are used to adjust, for example, the easiness of volume expansion of the receptor. The target of molecule designing of the present embodiment is the above-described polymer or the monomer unit constituting the polymer.

The polymer constituting the receptor is not particularly limited but examples thereof include silicone resin, acrylic resin, polyamide resin, polyurethane resin, cyanate resin, polyester resin, polyether resin, polyolefin resin, polystyrene resin, and polyphenylene oxide resin.

The filler is not particularly limited but may contain, for example, one or more selected from among polymer bead such as polystyrene, polymethyl methacrylate, or polyphenylene oxide, or spherical particle such as acrylic latex; metal nitride such as silicon nitride; metallic oxide such as silica, zirconia, titania, zinc oxide, aluminum oxide, or tin oxide; composite metallic compound such as barium titanate, strontium titanate, or ITO; inorganic metal filler such as gold, silver, copper, palladium, platinum, iron, or aluminum; and carbon material such as carbon nanotube, graphene, carbon black, carbon dot, or nanocarbon.

The receptor may include a void space. When the receptor includes a void space, it is possible to adjust a parameter related to physical strength or the like. Moreover, when the receptor includes a void space, an analyte is more likely to infiltrate into the receptor and, for example, the volume expansion rate can be adjusted. Accordingly, the sensitivity of the sensor can be adjusted.

1.2. Analyte

An analyte is a compound that mutually interacts the receptor and may be selected as appropriate in accordance with the purpose of a sensor to be produced. Such an analyte is not particularly limited but examples thereof include volatile organic compound (VOC) gas, oxidation-reduction gas, flammable gas, steam, and composite thereof.

2. Device

A device of the present embodiment comprises an estimation unit configured to input information A, information B, and reference information C to a model to output sensitivity information D of a receptor constituted by using a polymer for an analyte, the information A being related to the polymer constituting the receptor, the information B being related to the analyte, the reference information C being related to the film constitution of the receptor.

Accordingly, it is possible to quantify tacit knowledge and technological common knowledge of technical experts and obtain information for designing a molecule that can achieve performance required for a novel receptor. Moreover, it is possible to expect acceleration of material development and significant reduction of development cost.

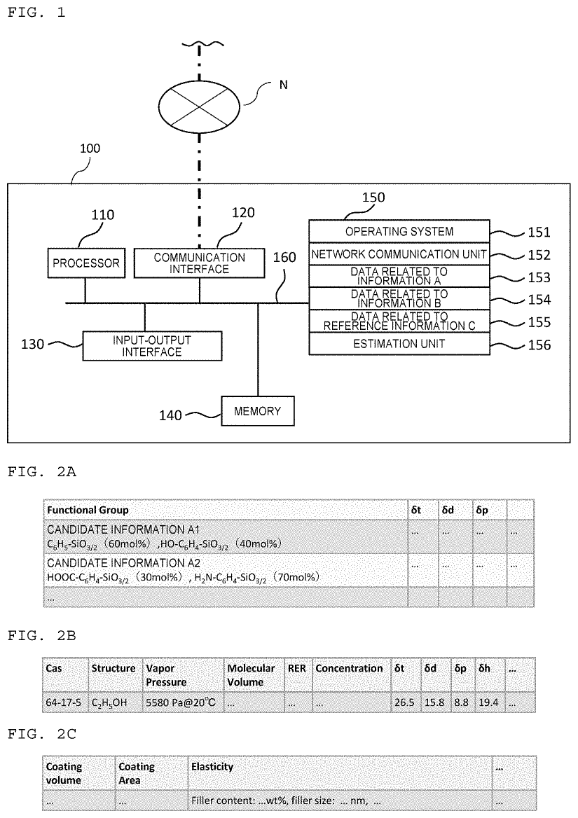

In the present embodiment, as illustrated in FIG. 1, for example, a device 100 may be connected to another device 200 through wired or wireless network N to acquire or output optional information. The device 100 may acquire at least part of information illustrated in FIG. 1 from another device such as a server connected through the network N or may execute, at another device such as a server connected through the network N, at least part of processing at functional components illustrated in FIG. 1.

2.1. Hardware Configuration

A hardware configuration of the device 100 will be described below with reference to FIG. 1. The device 100 includes, for example, a processor 110, a communication interface 120, an input-output interface 130, a memory 140, a storage 150, and one or a plurality of communication buses 160 for mutually connecting these constituent components.

The processor 110 executes a code included in a program stored in the storage 150 or a processing, function, or method implemented by a command. The processor 110 may include, as a non-limiting example, one or a plurality of central processing units (CPUs), micro processing units (MPUs), graphics processing units (GPUs), microprocessors, processor cores, multiprocessors, application-specific integrated circuits (ASICs), field programmable gate arrays (FPGAs), or the like and may implement processing, functions, or methods disclosed in each embodiment by using a logic circuit (hardware) or dedicated circuit formed in an integrated circuit (IC chip or large scale integration (LSI)) or the like.

The communication interface 120 transmits and receives various kinds of data to and from another device through the network. This communication may be executed in a wired or wireless manner and may use any communication protocol with which the communication is executable. For example, the communication interface 120 may be implemented with hardware such as a network adapter, communication software of any kind, or a combination thereof.

The network may be, as a non-limiting example, an ad hoc network, an intranet, an extranet, a virtual private network (VPN), a local area network (LAN), a wireless LAN (WLAN), a wide area network (WAN), a wireless WAN (WWAN), a metropolitan area network (MAN), part of the Internet, part of a public switched telephone network (PSTN), a cellular phone network, integrated service digital networks (ISDNs), wireless LANs, Long Term Evolution (LTE), Code Division Multiple Access (CDMA), Bluetooth (registered trademark), satellite communication, or a combination thereof. The network may include one or a plurality of networks.

The input-output interface 130 includes an input device configured to input various operations to the device 100, and an output device configured to output a processing result processed by the device 100. For example, the input-output interface 130 includes an information input device such as a keyboard, a mouse, or a touch panel, and an information output device such as a display. The device 100 may be connected to an external input-output interface 130 to receive certain inputting and execute certain outputting.

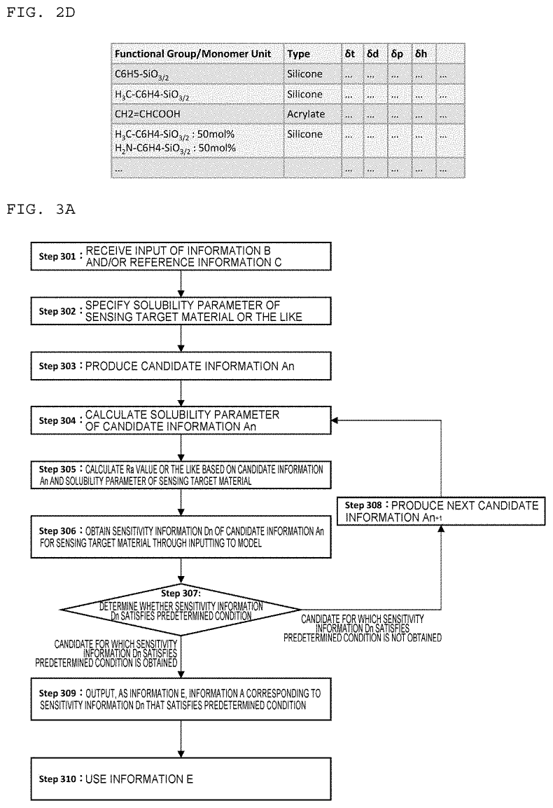

For example, the device 100 may be connected through the wired or wireless network N to the other device 200 as the external input-output interface 130 in which data (refer to FIG. 2D) of correspondence between the molecule structure of the polymer or the monomer unit and the SP value thereof is recorded. Accordingly, for example, the other device 200 maintains up-to-date the data of correspondence between the molecule structure of the polymer or the monomer unit and the SP value thereof so that the device 100 can constantly use the data maintained up-to-date. Thus, the device 100 can use a larger number of polymers and monomer units as the basis of estimation processing to be described later. Moreover, it is possible to reduce a waste of duplicate calculation and accumulation of the SP value by a plurality of individual devices 100. The other device 200 may be a server configured to provide the data of correspondence between the molecule structure of the polymer or the monomer unit and the SP value thereof in accordance with a request.

The term “molecule structure” means, for example, a functional group or molecular skeleton contained in the polymer or a molecule of the analyte, or the copolymerization ratio of the polymer.

The device 100 may be connected through the wired or wireless network N to the other device 200 as the external input-output interface 130 in which the information B related to the analyte is recorded. The information B related to the analyte may include a plurality of pieces of information such as vapor pressure, molecule volume, and a solubility parameter. The information is unique to the analyte and may be associated with a unique ID such as the Chemical Abstracts Service (CAS) number. Thus, when connected to the other device 200 in which the information B related to the analyte is recorded, the device 100 can immediately acquire the information B by using, for example, the CAS number as a main key. Accordingly, it is possible to reduce work of inputting the information B to the device 100 by a user. The other device 200 may be a server configured to provide the information B in accordance with a request.

The memory 140 temporarily stores a program loaded from the storage 150 and provides a work area to the processor 110. The memory 140 also temporarily stores various kinds of data generated while the processor 110 executes the program. The memory 140 may be a high-speed random access memory such as a DRAM, an SRAM, a DDR RAM, or any other random access solid storage device or may be a combination thereof.

The storage 150 stores programs, functional components, and various kinds of data. The storage 150 may be one or a plurality of nonvolatile memories such as magnetic disk storage devices, optical disk storage devices, flash memory devices, or any other nonvolatile solid storage devices or may be a combination thereof. Other examples of the storage 150 include one or a plurality of storage devices installed remotely from the processor 110.

In the embodiment of the present invention, the storage 150 stores programs, functional components, and data structures, or a subset thereof. The device 100 functions as an estimation unit 156 as illustrated in FIG. 1 when the processor 110 executes commands included in a program stored in the storage 150.

An operating system 151 processes, for example, various basic system services and includes procedures for executing tasks by using hardware.

A network communication unit 152 is used to, for example, connect the device 100 to another computer through the communication interface 120 and one or a plurality of communication networks such as the Internet, any other wide area network, a local area network, and a metropolitan area network.

Hereinafter, a “solubility parameter H1-1” or a “solubility parameter H2-1” means a one-dimensional Hildebrandt solubility parameter.

A “solubility parameter H1-2” or a “solubility parameter H2-2” means a three-dimensional solubility parameter of the London dispersion force term, the dipole term, and the hydrogen bond term. Examples of the three-dimensional solubility parameter may include the Hansen solubility parameter, the Van Krevelen-Hoftyzer solubility parameter, the Hoy SP value, and the Small SP value.

A “Hansen solubility parameter H1-3” or a “Hansen solubility parameter H2-3” means the Hansen solubility parameter among the above-described three-dimensional solubility parameters. The “Hansen solubility parameter” is also simply referred to as the “HSP value”.

The three-dimensional solubility parameter and the HSP value are lower-level concepts of the SP value in a broad sense and included in the SP value. In the present embodiment, the solubility parameter of the monomer unit constituting the receptor is referred to as a solubility parameter H1. The simple expression “solubility parameter H1” is used to mean a comprehensive concept of the solubility parameters H1-1 to H1-3 without distinction. The solubility parameter of the analyte is referred to as a solubility parameter H2. The simple expression “solubility parameter H2” is used to mean a comprehensive concept of the solubility parameters H2-1 to H2-3 without distinction. The “solubility parameter H1” and the “solubility parameter H2” are also referred to as “SP1” and “SP2”, respectively.

The simple expression “SP value” or “solubility parameter” is used to mean any of the above-described parameters without distinction.

2.1.1. Information A

Data 153 related to the information A stores information related to the solubility parameter H1 of the polymer constituting the receptor. The data 153 related to the information A may store the polymer constituting the receptor and SP1 thereof in association with each other. FIG. 2A illustrates an example of the data 153 related to the information A stored in the storage 150.

The information A is not particularly limited as long as it is any information related to SP1, and may be, for example, SP1 of the polymer or information for calculating SP1. The information for calculating SP1 is not particularly limited but examples thereof include the chemical formula (functional group) of the monomer unit. With the chemical formula of the monomer unit, it is possible to calculate SP1 by a group contribution method to be described later. Examples of chemical formula notation include SMILES, MOL, SDF, InChI, and InChIKey.

When the polymer constituting the receptor is a homopolymer, the data 153 related to the information A may store SP1 of one kind of a polymer, such as C₆H₅—SiO_{3/2}. When the polymer constituting the receptor is a copolymer, the data 153 related to the information A may store, for example, SP1 when two or more kinds of monomer units constituting the copolymer are used at a certain composition. Alternatively, an experimentally acquired measurement value may be used as SP1.

For example, when a copolymer using C₆H₅—SiO_{3/2} (60 mol %) and HO—C₆H₄—SiO_{3/2} (40 mol %) is assumed as illustrated in FIG. 2A, the data 153 related to the information A may store SP1 that can be derived by the group contribution method or the like based on the monomer-unit composition of C₆H₅—SiO_{3/2} (60 mol %) and HO—C₆H₄—SiO_{3/2} (40 mol %).

In the present embodiment, a “monomer unit” is a repeating unit constituting a polymer. For example, when silicone is assumed as the polymer, alkoxysilane is assumed as a monomer state before condensation. In a case with PhSi(OCH₃)₃ an example, which is alkoxysilane containing a phenyl radical, PhSi(OCH₃)₃ is a monomer and PhSiO_{3/2} is a monomer unit. In PhSiO_{3/2}, OCH₃ of PhSi(OCH₃)₃ becomes the bonding Si—O—Si through reaction.

In the present embodiment, the solubility parameter H1 may be calculated based on the molecule structure of a polymer or a monomer unit, or the molecule structure of a polymer or a monomer unit satisfying the solubility parameter H1 may be estimated based on the solubility parameter H1. In this case, a list of correspondence between a known monomer unit and the solubility parameter H1 of the monomer unit may be used. An example of such a list is illustrated in FIG. 2D.

As illustrated in FIG. 2D, the list may record the solubility parameter H1 for each polymer or monomer unit and may include the chemical formula (functional group) of a polymer or a monomer unit, the kind (type) of the monomer unit, or the like.

“Functional Group” may include information such as the chemical formula of a polymer or a monomer unit. The information such as the chemical formula may be used as information for uniquely specifying the polymer or the monomer unit.

“Type” may include information related to the kind of polymer constituted by a monomer unit such as Silicone or Acrylate as illustrated in FIG. 2D and may include, instead or in addition, information related to a polymerizable functional group. For example, when a polymer as a combination of two kinds of monomer units is suitable as a desirable receptor but the kinds of polymerizable functional groups of the monomer units are different from each other and copolymerization is impossible, it is impossible to synthesize the polymer as a combination of the two kinds of monomer units, and an output result is non-realistic. Thus, it may be determined whether copolymerization of monomer units is possible based on information stored in “Type”.

Accordingly, the structure of a polymer that can be actually synthesized can be output.

A solubility parameter is a value defined by regular solution theory introduced by Hildebrandt. Three-dimensional solubility parameters H are the London dispersion force term δd((MPa)^{1/2}), the dipole-dipole force term δp((MPa)^{1/2}), and the hydrogen bonding force term δh((MPa)^{1/2}) into which a solubility parameter δt((MPa)^{1/2}) is divided. Among them, a three-dimensional solubility parameter defined by Hansen is referred to as HSP. Such solubility parameters as three divided components can be treated as a three-dimensional vector, and it can be evaluated that compounds having similar vectors tend to have high affinity with each other. The accuracy of an output result tends to be further improved by using the three-dimensional solubility parameter.

Such a solubility parameter recorded in this manner may be calculated and stored in advance. For example, the data 153 related to the information A may be solubility parameters calculated and stored in advance for each of a finite number, for example, about 1000 of monomer units.

A parameter related to a solubility parameter H may be calculated based on a molecular dynamics calculation method, a method using machine learning or deep learning, evaporative latent heat, or cohesion energy density. The calculation is not particularly limited to but, for example, may be performed by a relatively simple group contribution method. The group contribution method is a method of assuming that a physical property depends on the number and kind of functional groups (groups) such as —CH₃ or —OH, dividing the structure of a material into groups, and estimating the physical property by using parameters allocated to the groups. The group contribution method of three-dimensional solubility parameters is not particularly limited but examples thereof include a Hoy method (K. L. Hoy, J. Paint Techn., 1970, 42, 76), a van Krevelen-Hoftyzer method (Van Krevelen, D. W., Hoftyzer, P. J. J. Appl. Polymer Sci. 1967, 11, 2189), a method disclosed in Japanese Patent Laid-open No. 2018-173336, and in particular, examples of a method of calculating the Hansen solubility parameter include a YMB simulator incorporated in HSPiP software (https://www.hansen-solubility.com/HSPiP/) and a Stefanis-Panayioyou method (Stefanis E., Panayiotou C. International Journal of Thermophysique, 2008, 29, 568).

When experimentally determined, the solubility parameter H1 may be determined from physical quantities, for example, surface tension for δt, a refractive index for δd, a dielectric constant and dipole-dipole force for δp, and a solvent polarity parameter for δh or may be determined by using the solubility sphere method, the iGC method, or the like based on various kinds of solvent solubility tests.

2.1.2. Information B

The information B related to the analyte is information related to the analyte that mutually interacts the receptor. FIG. 2B illustrates an example of data 154 related to the information B stored in the storage 150. As illustrated in FIG. 2B, the information B may include the solubility parameter H2, molecule structure, Cas number, molecule volume, saturated vapor pressure, relative volatility (PER), concentration, and the like, of the analyte.

In FIG. 2B, the Cas number is a value identification number that is unique to a chemical material and provided by a chemical material registration system operated and managed by the chemical abstracts service (CAS). The Cas number may be used as a unique ID for the system to uniquely recognize the analyte.

The solubility parameter H2 of the analyte may include the solubility parameter δt((MPa)^{1/2}), the London dispersion force term δd((MPa)^{1/2}), the dipole-dipole force term δp((MPa)^{1/2}), and the hydrogen bonding force term δh((MPa)^{1/2}). When the information B includes the solubility parameter H2 of the analyte, affinity with the solubility parameter H1 in the information A can be taken into consideration.

The concentration is an assumed concentration of the analyte in an atmosphere in which the receptor and the analyte contact each other. The concentration of the analyte changes in accordance with usage. Thus, when such an assumed concentration is taken into consideration, the receptor can be designed based on more appropriate consideration of mutual interaction between the receptor and the analyte.

2.1.3. Reference information C

As described above, in the receptor, the polymer adsorbs the analyte and expands volume, and the degree of the volume expansion is adjusted by the film constitution such as the ratio of fillers and void spaces or the volume of the receptor. Thus, as the film constitution of the receptor changes, a physical parameter such as the volume expansion rate of the receptor changes and the sensitivity information D is affected. The sensor of the present embodiment detects such change of the receptor by the sensor body, and thus, in order to assume such change of the receptor and design a more appropriate sensor, it is desirable to take into consideration any other element that affects a physical parameter of the receptor other than the polymer, such as the profile, filler, and void space of a receptor to be formed.

The reference information C includes such information related to the film constitution of the receptor. Specifically, as illustrated in FIG. 2C, the reference information C may include: information related to the profile of the receptor, such as film volume, film area, and film shape; and information related to hardness and the like, of physical parameters such as the kind of the filler, the composition amount of the filler, the particle diameter of the filler, and the void space ratio.

2.1.4. Estimation Unit

The estimation unit 156 executes a sensitivity estimation step of inputting the information A, the information B, and the reference information C to the model to output the sensitivity information D of the receptor constituted by using the polymer for the analyte, the information A being related to the solubility parameter H1 of the polymer constituting the receptor, the information B being related to the analyte, the reference information C being related to the film constitution of the receptor.

The sensitivity information D is information related to sensitivity of the receptor for the analyte and may include information related to volume change of the receptor at mutual interaction between the analyte and the receptor. The sensitivity information D is not particularly limited but examples thereof include the amplitude strength of a signal, the differential strength or integral strength of the signal, the rising time or falling time of the signal, and these time constants and the like as numerical values representing the degree of the volume expansion rate when the analyte at a certain concentration is adsorbed at a certain temperature. However, the sensitivity information D is not limited thereto. Since a physical parameter detected as sensitivity differs depending on the kind of the sensor, the sensitivity information D may be selected as appropriate depending on the kind of the sensor.

In the sensitivity estimation step, the estimation unit 156 may additionally input information F to the model to output the sensitivity information D of the receptor constituted by using the polymer for the analyte, the information F being related to mutual interaction between the polymer and the analyte. Accordingly, it is possible to select a monomer unit that performs certain mutually interaction with the analyte.

The information F may include a x parameter and/or a Ra parameter. The x parameter is a mutual interaction parameter in Flory-Huggins theory and a dimensionless parameter that means mutual interaction and is calculated based on change of polymer-low-molecule combined free energy in a lattice model. The x parameter is calculated by a formula below.

$$χ = ( \frac{{V}_{1}}{R T} ) {( {δ}_{1} - {δ}_{2} )}^{2} \qquad [ Expression 1 ]$$

χ: χ parameter
δ₁: solubility parameter of solvent
δ₂: solubility parameter of unknown specimen solvent
V₁: mole volume of solvent
R: gas constant
T measurement temperature

The Ra parameter is a parameter representing the inter-vector distance between three-dimensional solubility parameters H1 including the Hansen solubility parameter and three-dimensional solubility parameters H2 including the Hansen solubility parameter. Ra is calculated by a formula below.

Rₐ=[4(δ_{d1}−δ_{d2})²+(δₚ₁−δₚ₂)²+(δₕ₁−δₕ₂)²]^{1/2} [Expression 2]
Rₐ [MPa^{1/2}]· ·HSP distance between two components
δ_{d}: London dispersion force term [(MPa)^{1/2}]
δₚ: dipole-dipole force term [(MPa)^{1/2}]
δₕ: hydrogen bonding force term [(MPa)^{1/2}]

In the sensitivity estimation step, the estimation unit 156 may additionally output, from the model, recommendation information C′ related to the film constitution of the receptor. The recommendation information C′ is not particularly limited but may be, for example, recommendation information of what film constitution the receptor constituted by using the polymer needs to have to produce a desired sensor. In this manner, it is possible to not only simply propose a monomer unit having certain sensitivity information D but also propose the film constitution of the receptor produced by using the monomer unit, with which a polymer designed by selecting the monomer unit more appropriately functions, by proposing the recommendation information C′ such as the filler amount or void space amount of the receptor.

The recommendation information C′ includes information related to the film constitution of the receptor, and specifically, may include information related to the profile of the receptor, such as film volume, film area, and film shape; and information related to hardness and the like, of physical parameters such as the elastic modulus of the receptor, the composition amount of the filler, the particle diameter of the filler, the void space ratio.

2.1.5. Model

Input values of the model used in the present embodiment are the information A, the information B, and the reference information C, and an output value of the model is the sensitivity information D of the receptor constituted by using the polymer for the analyte. In addition, the model used in the present embodiment may include, as an input value, the information F related to mutual interaction between the polymer and the analyte.

The model used in the present embodiment may include, as output values, information E related to the molecule structure of the polymer that satisfies the sensitivity information D, and the recommendation information C′ related to the film constitution of the receptor.

Such a model may be a learning-completed model obtained by performing machine learning processing based on learning data including the above-described input values and the above-described output values. The machine learning is not limited to a particular algorithm but may use a well-known learning algorithm such as PLS, ridge regression, Lasso regression, decision tree, ensemble learning including gradient boosting and random forest, support vector machine, or neural network (NN).

3. Method (Operation Processing)

Next, a method of operation processing at a device according to the present embodiment will be described below. The method of the present embodiment executes the sensitivity estimation step in which the device inputs the information A, the information B, and the reference information C to the model to output the sensitivity information D of the receptor constituted by using the polymer for the analyte, the information A being related to SP1 of the polymer constituting the receptor, the information B being related to the analyte, the reference information C being related to the film constitution of the receptor.

The method according to the present embodiment can be divided into procedures of a molecular-skeleton-first method and a SP-first method.

3.1. Molecular-Skeleton-First Method

In the molecular-skeleton-first method, a composition candidate of the monomer unit of the polymer constituting the receptor, in other words, a candidate of the molecular skeleton of the polymer is specified first as the information A. Then, the sensitivity information D when the receptor is formed by using the polymer having the molecular skeleton is estimated.

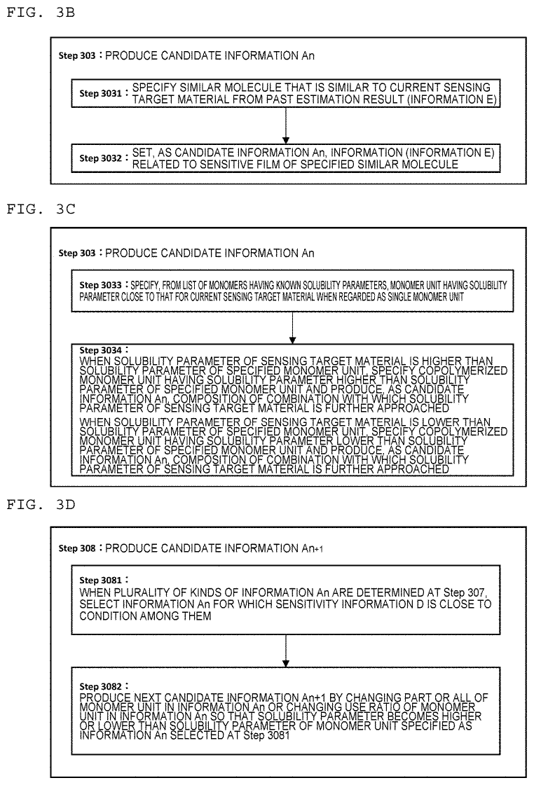

A processing method of the molecular-skeleton-first method will be described below with reference to FIGS. 3A to 3D.

3.1.1. Input of Information B and Reference Information C

At Step S301, the input-output interface 130 of the device 100 may receive input of the information B related to the analyte and/or the reference information C related to the film constitution of the receptor. The estimation unit 156 of the device 100 may store the received pieces of information in the data 154 and 155, respectively.

At Step S302, the estimation unit 156 of the device 100 may specify the solubility parameter H2 of the analyte based on the information B. In this case, the estimation unit 156 of the device 100 may calculate the solubility parameter H2 by the group contribution method.

3.1.2. Processing of Producing Candidate Information a

At Step S303, the estimation unit 156 of the device 100 may produce the information A related to the polymer constituting the receptor. In the molecular-skeleton-first method, a composition candidate of the monomer unit of the polymer constituting the receptor, in other words, one or a plurality of candidates of the molecular skeleton of the polymer may be specified first as the information A. According to the group contribution method, SP1 can be estimated based on the molecule structure of the polymer or monomer unit constituting the receptor. Thus, the information A produced here may include, in place of or in addition to SP1, information related to the molecule structure of the polymer or monomer unit constituting the receptor.

In the molecular-skeleton-first method, at production of the information A, an appropriate monomer unit may be selected from the list of correspondence between a known monomer unit and the solubility parameter H1 of the monomer unit. The monomer units included in the list may be those having structures known to a technical expert, such as those attributable to a commercially available monomer and those attributable to a monomer used by the technical expert in the past. Thus, in the molecular-skeleton-first method, a monomer unit suitable for the analyte is selected from among monomer units that can originally be synthesized to some extent, and accordingly, the realizability of designed molecule synthesis is excellent.

The information A produced at Step S303 may be a candidate of the information E to be output in a later step. Specifically, in a later step, the estimation unit 156 outputs the sensitivity information D for the analyte in accordance with the produced information A. Then, when the sensitivity information D satisfies a predetermined condition, the estimation unit 156 can output the information A as the information E. On the other hand, when the sensitivity information D does not satisfy the predetermined condition, the estimation unit 156 can produce new information A in place of the information A and output the sensitivity information D in accordance with the new information A. In this manner, the estimation unit 156 can execute loops of steps S303 to S307 until the sensitivity information D for the produced information A satisfies the predetermined condition.

In FIGS. 3A to 3D, indexes “n” and “n+1” are attached to the information A and the sensitivity information D. Information An means the information A produced in the n-th loop, and sensitivity information Dn means the sensitivity information D output based on the information An. Information An+1 means the information A produced in the (n+1)-th loop, and sensitivity information Dn+1 means the sensitivity information D output based on the information An+1.

At Step S303, the estimation unit 156 may produce the information A by randomly selecting the polymer or monomer unit constituting the receptor or in accordance with a certain algorithm. FIGS. 3C and 3D illustrate an example of the method of producing the information A.

3.1.2.1. Example 1 of Processing of Producing Candidate Information a

For example, in FIG. 3B, the estimation unit 156 produces the information A by using a result of estimation performed in the past. In this method, at Step S3031, the estimation unit 156 specifies a similar molecule that is similar to the current analyte from the information E related to a polymer molecule structure estimated for another analyte. The method of similar molecule specification is not particularly limited but examples thereof include a method of using the SP value as a reference and specifying a molecule having an SP value close to the SP value of the current analyte by using the x parameter, the Ra parameter, or the like.

Subsequently at Step S3032, the estimation unit 156 sets, as candidate information A, the information E related to a polymer molecule structure estimated for the specified similar molecule. Specifically, the information E related to the polymer molecule structure estimated for the specified similar molecule is used as candidate information A based on provisional estimation that a receptor molecule structure suitable for the similar molecule is also suitable as a receptor molecule structure for the current analyte.

Accordingly, it is possible to simply and appropriately produce the information A by using a result of estimation performed in the past. One molecule having the closest SP value or one or a plurality of similar molecules that satisfy a certain x parameter or Ra parameter with the current analyte may be selected as the specified similar molecule. When a plurality of similar molecules are specified, a plurality of pieces of the candidate information A can be produced. Accordingly, a plurality of pieces of sensitivity information D can be obtained for the plurality of pieces of information A, respectively, and information A having more appropriate sensitivity information D can be specified from among them.

3.1.2.2. Example 2 of Processing of Producing Candidate Information a

As another example, in FIG. 3C, for example, the estimation unit 156 produces the information A by using a list of correspondence between a known monomer unit and the SP value of the monomer unit and the group contribution method. In this method, at Step S3033, the estimation unit 156 specifies, from the list of correspondence between a known monomer unit and the SP value of the monomer unit, a monomer unit having an SP value close to that for the current analyte when regarded as a single monomer unit.

Subsequently at Step S3034, the estimation unit 156 selects another monomer unit to be copolymerized so that the SP value of the specified monomer unit approaches the SP value of the analyte, and produces the composition of the combination of those monomer units as candidate information A. More specifically, when the SP value of the analyte is higher than the SP value of the specified monomer unit, a copolymerized monomer unit having an SP value higher than the SP value of the specified monomer unit is specified and the composition of the combination with which the SP value of the analyte is further approached is produced as candidate information A; and when the SP value of the analyte is lower than the SP value of the specified monomer unit, a copolymerized monomer unit having an SP value lower than the SP value of the specified monomer unit is specified and the composition of the combination with which the SP value of the analyte is further approached is produced as candidate information A.

Accordingly, without past information, it is possible to simply and appropriately produce the information A by using the list of correspondence between a known monomer unit and the SP value of the monomer unit. One monomer unit having the closest SP value or one or a plurality of monomer units that satisfy a certain x parameter or Ra parameter with the current analyte may be selected as the monomer unit specified at Step S3033. When a plurality of monomer units are specified, a plurality of pieces of the candidate information A can be produced. Accordingly, a plurality of pieces of sensitivity information D can be obtained for the plurality of pieces of information A, respectively, and information A having more appropriate sensitivity information D can be specified from among them.

3.1.3. Processing of Calculating Information F Related to Mutual Interaction Between Receptor and Analyte

As described above, when a molecular skeleton is specified first as candidate information A, the estimation unit 156 of the device 100 may calculate SP1 of the candidate information A by the group contribution method or the like based on information related to the molecule structure of the polymer at Step S304.

At Step S305, the estimation unit 156 of the device 100 may output the information F related to mutual interaction between the receptor and the analyte based on SP1 of the candidate information A and SP2 of the analyte. Here, the information F related to the mutual interaction is not particularly limited but the x parameter, the Ra value, or the like may be calculated. The x parameter calculated here, the Ra parameter calculated here, or the like may be used as one piece of information to be input to the model in the following estimation processing.

Instead of additionally inputting the information F to the model, the estimation unit 156 of the device 100 may consider SP1 of the information A and SP2 of the information B in the model at Step S306 to be described later. More specifically, the estimation unit 156 of the device 100 may calculate the x parameter and/or the Ra parameter based on SP1 of the information A and SP2 of the information B and consider the calculated value in the model. Accordingly, it is possible to select a polymer having certain sensitivity for the analyte and select a monomer unit constituting the polymer.

3.1.4. Estimation Processing

At Step S306, the estimation unit 156 of the device 100 inputs the information A produced as described above, the information B related to the analyte, and the reference information C related to the film constitution of the receptor to the model to output the sensitivity information D of the receptor constituted based on the information A for the analyte. This step is also referred to as the sensitivity estimation step.

The model is not particularly limited but may be, for example, a learning-completed model generated by machine learning processing based on learning data. The learning data may include the information A related to SP1 of the polymer constituting the receptor, the information B related to the analyte, the reference information C related to the film constitution of the receptor, and the sensitivity information D of the receptor constituted by using the polymer for the analyte. In other words, the learning data represents the degree of sensitivity when a certain analyte interacts with a receptor specified by a certain polymer and film constitution.

The model generated by machine learning processing with a sufficient amount of such learning data outputs the sensitivity information D of the receptor for the analyte based on information (the information A and C) related to the polymer constituting the receptor and the film constitution thereof and the information B related to the analyte.

In the sensitivity estimation step, the estimation unit 156 of the device 100 may additionally output the recommendation information C′ related to the film constitution of the receptor in addition to the sensitivity information D by using the model. Physical characteristics of the receptor can be affected by the shape of the receptor such as volume, the filler ratio, and the void space ratio even when the same polymer is used. Taking into consideration such influence, the estimation unit 156 of the device 100 can provide the sensitivity information D of the polymer and also suggest what filler contained amount and void space amount the receptor needs to have when the receptor is produced by using the polymer having a certain structure.

At Step S307, the estimation unit 156 of the device 100 may determine whether the sensitivity information D satisfies a predetermined condition. The predetermined condition is not particularly limited but examples thereof include a condition that the sensitivity information D is equal to or larger than a certain threshold value; a condition that a local maximum value or a local minimum value is obtained by optimization processing with the sensitivity information D as an objective function; and a condition related to the sensitivity information D determined in advance as necessary.

3.1.5. Case in which Sensitivity Information D does not Satisfy Predetermined Condition (Loop Processing)

When the sensitivity information D does not satisfy the predetermined condition, the estimation unit 156 of the device 100 may produce next candidate information An+1 at Step S308, again. The information An+1 may be produced by using the method described at Step S303 or based on the information An. When loop processing is performed in this manner, a local maximum value or local minimum value of the objective function of the sensitivity information D can be searched by checking the sensitivity information Dn, Dn+1, Dn+2, . . . for the information An, An+1, An+2, . . . and optimization processing can be performed.

FIG. 3D illustrates an example of the method of producing the information An+1 based on the information An. In this method, for example, when a plurality of kinds of the information An are determined at Step S307, the estimation unit 156 selects the information An for which the sensitivity information D is close to the above-described predetermined condition among the plurality of kinds of the information An at Step S3081. Step S3081 may be omitted when one kind of the information An is determined at Step S307.

Subsequently at Step S3082, the estimation unit 156 produces next candidate information An+1 by changing part or all of the monomer unit in the information An or changing the use ratio of the monomer unit in the information An so that the SP value becomes higher or lower than the SP value of the polymer specified as the information An selected at Step S3081. Accordingly, one or a plurality of pieces of the information An+1 for which the composition of the monomer unit is similar to that for the information An can be produced. The information A for which the sensitivity information D is more closer to the above-described predetermined condition can be searched by executing steps S304 to S306 again for such information An+1 to output the sensitivity information Dn+1.

Step S308 will be described below with a specific example. For example, when the sensitivity information D is acquired as information A1 and A2 for two monomer unit compositions of Copolymers 1 and 2 below, the sensitivity information D does not satisfy the predetermined condition. Then, at Step S3081, the estimation unit 156 selects a copolymer for which the sensitivity information D is closer to a condition determined in advance among Copolymers 1 and 2. In this example, it is assumed that Copolymer 1 is selected.

Information A1: Copolymer 1: C₆H₅—SiO_{3/2} (60 mol %), HO—C6H4-SiO_{3/2} (40 mol %)

Information A2: Copolymer 2: HOOC—C₆H₄—SiO_{3/2} (30 mol %), H2N—C6H4-SiO3/2 (70 mol %)

Then, at Step S3081, the estimation unit 156 changes the monomer-unit ratio of Copolymer 1 and produces Copolymers 3 and 4 as next candidate information An+1. Then, the information A including more appropriate sensitivity information D can be searched by executing steps S304 to S306 again for such information An+1 to output the sensitivity information Dn+1.

Information A3: Copolymer 3: C₆H₅—SiO_{3/2} (55 mol %), HO—C6H4-SiO3/2 (45 mol %)

Information A4: Copolymer 4: C₆H₅—SiO_{3/2} (65 mol %), HO—C6H4-SiO3/2 (35 mol %)

What is to be selected as candidate information An+1 or how much the information An is to be changed to produce the information An+1 may be determined by using a well-known algorithm such as a genetic algorithm, a bootstrap method, or Bayesian optimization.

In the above description, the new information An+1 is produced by changing the information An, but at Step S308, a condition in the information C in addition to the information A may be changed to execute steps S304 to S306 again. For example, more appropriate sensitivity information D may be obtained by changing, as a condition included in the information C, the filler kind or contained amount, the void space ratio, the film volume, or the like to adjust the hardness, flexibility, or volume change easiness of the film.

3.1.6. Case in which Sensitivity Information D Satisfies Predetermined Condition (Output Processing)

When the sensitivity information D satisfies the predetermined condition, the estimation unit 156 of the device 100 may additionally output the information E related to the monomer molecule structure based on the sensitivity information D at Step S309 (molecule structure estimation process). In this case, the estimation unit 156 may output, as the information E, the information A corresponding to the sensitivity information Dn that satisfies the predetermined condition. The information E related to the molecule structure of the polymer of the receptor may be the molecule structure of the polymer or the monomer unit constituting the polymer in the information A corresponding to the sensitivity information Dn that satisfies the predetermined condition. The information E may further include information related to the film constitution of the receptor, which is preferable for satisfying predetermined sensitivity information D, such as the filler ratio, the void space ratio, or the film constitution such as the volume of the receptor.

3.1.7. Use of Information E

At Step S310, the estimation unit 156 of the device 100 may output the information E to the input-output interface 130. More specifically, the estimation unit 156 of the device 100 may output and control the information E on a display or may output the information E to another device.

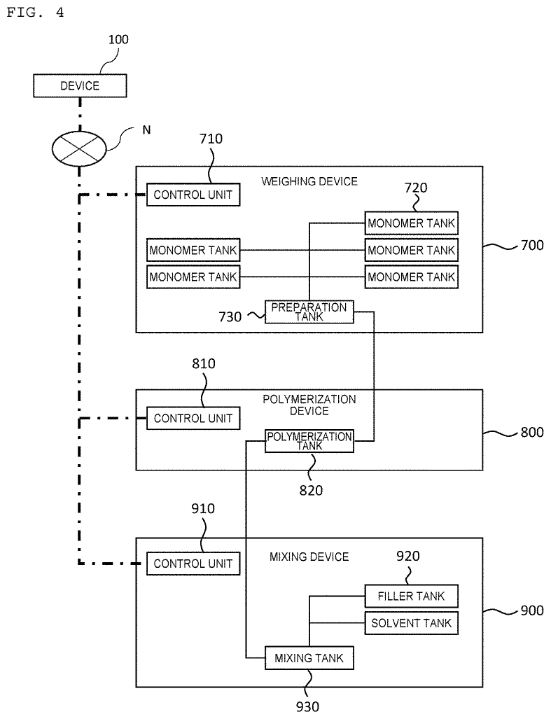

FIG. 4 illustrates an aspect of a case in which the information E is output to other devices. FIG. 4 illustrates a weighing device 700, a polymerization device 800, and a mixing device 900 as the other devices.

The estimation unit 156 of the device 100 may output the information E to the weighing device 700. A control unit 710 of the weighing device 700 can weigh each monomer based on the information E and adjust the material of the receptor. The weighing device 700 may include tanks 720 in which the respective monomers are stored and a preparation tank 730 connected to those tanks through pipes or the like. For example, when the information E is information related to the receptor in which 50 units of a monomer α and 50 units of a monomer β are copolymerized, the weighing device 700 can weigh 50 units of each monomer from a tank of the monomer a and a tank of the monomer γ, respectively, and guide each weighed monomer to the preparation tank 730.

In this manner, the information E may be transmitted to and used at such a weighing device configured to adjust the material of the receptor.

The weighing device 700 may be connected to the polymerization device 800 configured to polymerize the material of the receptor, which is adjusted as described above, or the weighing device 700 may be part of the polymerization device 800. A polymerization tank 820 of the polymerization device 800 polymerizes the material of the receptor, which is adjusted as described above, under control of a polymerization condition by a control unit 810.

A control unit 910 of the mixing device 900 can weigh and mix a filler or the like with the polymer based on the information E. The mixing device 900 may include other tanks 920 in which fillers and solvents other than monomers are stored and a mixing tank 930 connected to the tanks through pipes or the like. The fillers may be stored in individual tanks for each material kind and each particle diameter. For example, when the information E is information related to a receptor containing a filler having a certain particle diameter, the mixing device 900 can weigh the certain filler and guide the weighed filler to the mixing tank 930. Accordingly, a receptor formation composition is obtained in the mixing tank 930.

Note that the other devices are not limited to the above-described configuration. For example, the weighing device 700 may further include another tank in which, for example, a filler or a solvent other than a monomer is stored, and the polymerization device 800 may polymerize each monomer under existence of the filler or the like. In this case, no mixing device 900 may be provided.

3.2. SP-First Method

The SP-first method will be described below. In the molecular-skeleton-first method, a candidate for the molecule structure of a polymer or a monomer unit is produced in advance and the sensitivity information of the polymer is estimated, but in the SP-first method, a candidate for the SP value is produced in advance and the sensitivity information D is estimated based on the SP value to specify an optimum SP value. Then, a polymer structure that satisfies the specified optimum SP value is specified by, for example, the group contribution method or the like. In other words, a candidate for a polymer molecule structure is produced first in the molecular-skeleton-first method, but a molecule structure is specified after an SP value is specified in the SP-first method. Thus, in the molecular-skeleton-first method, a known molecule structure of a polymer or a monomer unit needs to be considered to first produce a candidate for the polymer molecule structure. However, in the SP-first method, an optimum SP value is specified without specifying a molecule structure, and thereafter the structure is estimated based on the optimum SP value, and thus a monomer unit structure that is unknown to a technical expert can be considered.

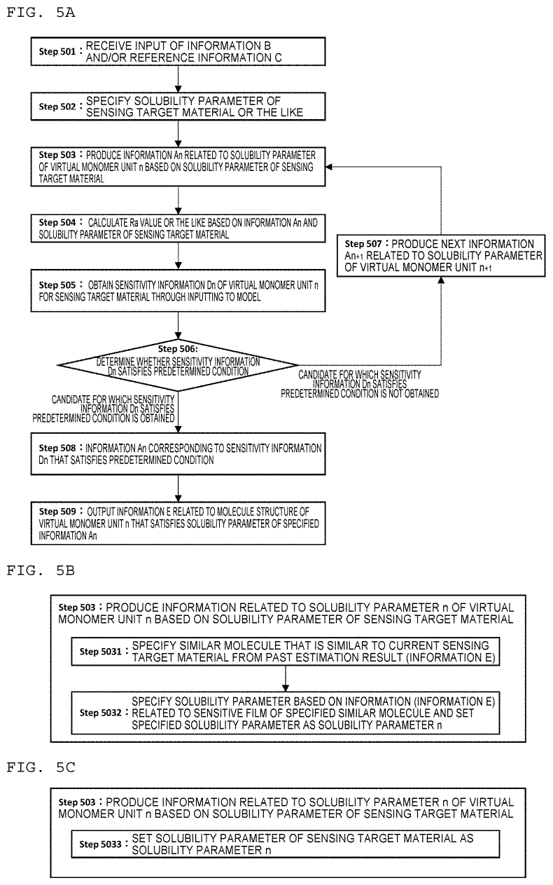

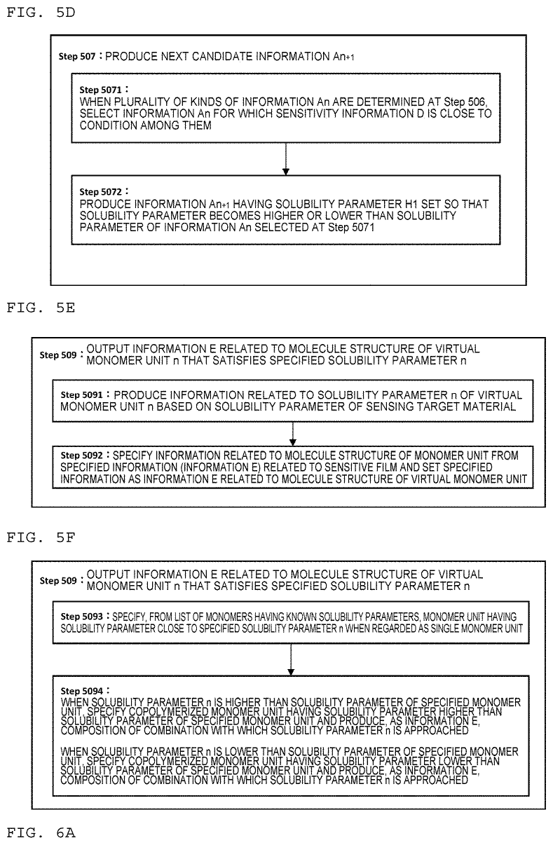

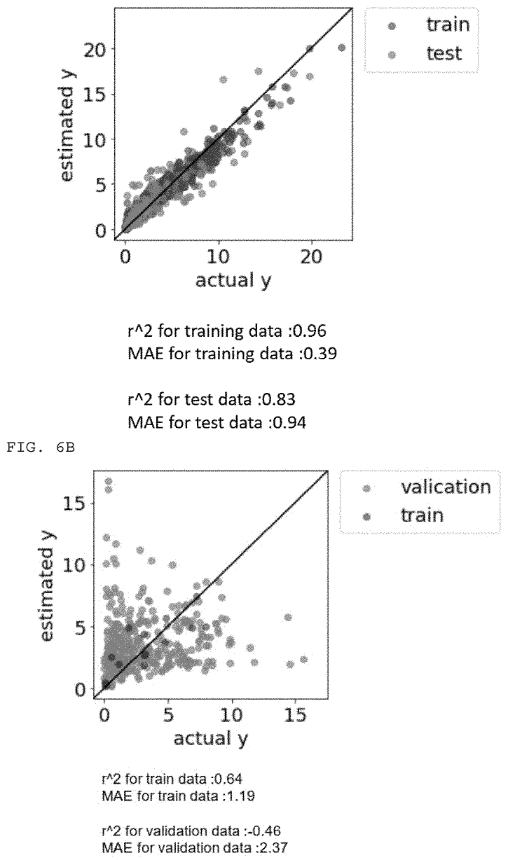

A processing method of the SP-first method will be described below with reference to FIGS. 5A to 5E.

3.2.1. Input of Information B and Reference Information C

At Step S501, the input-output interface 130 of the device 100 may receive input of the information B related to the analyte and/or the reference information C related to the film constitution of the receptor. The estimation unit 156 of the device 100 may store the received pieces of information in the data 154 and 155, respectively.

At Step S502, the estimation unit 156 of the device 100 may specify the solubility parameter H2 of the analyte based on the information B. In this case, the estimation unit 156 of the device 100 may calculate the solubility parameter H2 by the group contribution method.

3.2.2. Processing of Producing Candidate Information a

At Step S503, the estimation unit 156 of the device 100 may produce the information A related to the solubility parameter H1 of the polymer constituting the receptor. In the SP-first method, the SP value of the polymer constituting the receptor is specified first as the information A. Specifically, at a particular time point of the SP value of the polymer, the information A may include no information related to a specific molecular skeleton corresponding to the SP value.

In this manner, in the SP-first method, the SP value is specified and the processing of estimating the sensitivity information D proceeds without specifying a specific structure of the polymer or the monomer unit, and then processing of specifying a specific structure of the polymer or the monomer unit based on the SP value is performed. For sake of simplicity, the SP value of a polymer for which no specific structure is specified is referred to as the “SP value of a virtual polymer”. In addition, for sake of simplicity, a monomer unit for which no specific structure is yet to be specified is referred to as a “virtual monomer unit”.

The information A produced at Step S503 may be a candidate for the information E to be output in a later step. Specifically, in a later step, the estimation unit 156 outputs the sensitivity information D for the analyte in accordance with the produced information A. Then, when the sensitivity information D satisfies the predetermined condition, the estimation unit 156 can generate a molecule structure that satisfies the information A and output the molecule structure as the information E. On the other hand, when the sensitivity information D does not satisfy the predetermined condition, the estimation unit 156 can produce new information A in place of the information A and output the sensitivity information D in accordance with the new information A. In this manner, the estimation unit 156 can execute loops of steps S504 to S508 until the sensitivity information D for the produced information A satisfies the predetermined condition.

At Step S503, the estimation unit 156 may randomly produce SP1 included in the information A or in accordance with a certain algorithm. FIGS. 5C and 5D illustrate an example of the method of producing the information A.

3.2.2.1. Example 1 of Processing of Producing Candidate Information a

For example, in FIG. 5B, the information A is produced by using a result of estimation performed in the past. In this method, at Step S5031, the estimation unit 156 specifies a similar molecule that is similar to the current analyte from the information E related to a polymer molecule structure estimated for another analyte. The method of similar molecule specification is not particularly limited but examples thereof include a method of using the SP value as a reference and specifying a molecule having an SP value close to the SP value of the current analyte.

Subsequently at Step S5032, the estimation unit 156 sets, as candidate information A, the information E related to a polymer molecule structure estimated for the specified similar molecule. Specifically, the SP value of the information E related to the polymer molecule structure estimated for the specified similar molecule is used as candidate information A based on provisional estimation that a receptor molecule structure suitable for the similar molecule is also suitable as a receptor molecule structure for the current analyte.

Accordingly, it is possible to simply and appropriately produce the information A by using a result of estimation performed in the past. One molecule having the closest SP value or one or a plurality of similar molecules that satisfy a certain x parameter or Ra parameter with the current analyte may be selected as the specified similar molecule. When a plurality of similar molecules are specified, a plurality of pieces of the candidate information A can be produced. Accordingly, a plurality of pieces of sensitivity information D can be obtained for the plurality of pieces of information A, respectively, and information A having more appropriate sensitivity information D can be specified from among them.

3.2.2.2. Example 2 of Processing of Producing Candidate Information a

As another example, in FIG. 5C, for example, the information A is produced by using SP2 of the analyte. In this method, at Step S3033, the estimation unit 156 may calculate SP2 of the current analyte and specify SP1 of the same value as the information A.

The sensitivity information D is, for example, sensitivity obtained by detecting change of a physical parameter such as the volume change rate due to interaction of the analyte with the receptor. Thus, matching between SP1 and SP2 does not allow determination that the sensitivity information D is excellent, but the sensitivity information D is estimated based on consideration of both the information A and the reference information C.

Thus, as illustrated in FIG. 5C, even when SP1 of the same value as SP2 is specified as the information A, this information A is not directly an optimum solution but the sensitivity information D that satisfies the predetermined condition is specified through steps described below and the information E is estimated based on the specified sensitivity information D.

3.2.3. Processing of Calculating Information F Related to Mutual Interaction Between Receptor and Analyte

At Step S504, the estimation unit 156 of the device 100 may output, based on SP1 of the candidate information A and SP2 of the analyte, the information F related to mutual interaction between the receptor and the analyte. The information F related to the mutual interaction is not particularly limited but the x parameter, the Ra value, or the like may be calculated. The calculated x parameter, the calculated Ra parameter, or the like may be used as one piece of information to be input to the model in the following estimation processing.

Instead of additionally inputting the information F to the model, the estimation unit 156 of the device 100 may consider SP1 of the candidate information A and SP2 of the analyte in the model at Step S505 to be described later. More specifically, the estimation unit 156 of the device 100 may calculate the x parameter and/or the Ra parameter based on SP1 of the candidate information A and SP2 of the analyte and consider the calculated value in the model. Accordingly, it is possible to select a polymer having certain sensitivity for the analyte.

3.2.4. Estimation Processing

At Step S505, the estimation unit 156 of the device 100 inputs the information A produced as described above, the information B related to the analyte, and the reference information C related to the film constitution of the receptor to the model to output the sensitivity information D of the receptor constituted based on the information A for the analyte. This step is also referred to as the sensitivity estimation step.

The model is not particularly limited but may be the model described in the molecular-skeleton-first method.

In the sensitivity estimation step, the estimation unit 156 of the device 100 may additionally output the recommendation information C′ related to the film constitution of the receptor in addition to the sensitivity information D by using the model. Physical characteristics of the receptor can be affected by the shape of the receptor such as volume, the filler ratio, and the void space ratio even when the same polymer is used. Taking into account such influence, the estimation unit 156 of the device 100 can provide the sensitivity information D of the polymer and also what filler contained amount and void space amount the receptor needs to have when the receptor is produced by using the polymer having a certain structure.

At Step S506, the estimation unit 156 of the device 100 may determine whether or not the sensitivity information D satisfies the predetermined condition. The predetermined condition is not particularly limited but examples thereof include a condition that the sensitivity information D is equal to or larger than a certain threshold value; a condition that a local maximum value or a local minimum value is obtained by optimization processing with the sensitivity information D as an objective function; and a condition related to the sensitivity information D determined in advance as necessary.

3.2.5. Case in which Sensitivity Information D does not Satisfy Predetermined Condition (Loop Processing)

When the sensitivity information D does not satisfy the predetermined condition, the estimation unit 156 of the device 100 may produce next candidate information An+1 at Step S507. The information An+1 may be produced by using the method described at Step S503 or based on the information An. When loop processing is performed in this manner, a local maximum value or local minimum value of the objective function of the sensitivity information D can be searched by checking the sensitivity information Dn, Dn+1, Dn+2, . . . for the information An, An+1, An+2, . . . and optimization processing can be performed.

FIG. 5D illustrates an example of the method of producing the information An+1 based on the information An. In this method, for example, when a plurality of kinds of the information An are determined at Step 506, the estimation unit 156 selects the information An for which the sensitivity information D is close to the above-described predetermined condition among the plurality of kinds of the information An at Step S071. Step S071 may be omitted when one kind of the information An is determined at Step 506.

Subsequently at Step S072, the estimation unit 156 produces next candidate information An+1 having SP1 set so that the SP value becomes higher or lower than SP1 of the information An selected at Step S071. Accordingly, one or a plurality of the information An+1 for which the composition of the polymer or the monomer unit is similar to that for the information An can be produced. The information A for which the sensitivity information D is closer to the above-described predetermined condition can be searched by executing steps S503 to S505 again for such information An+1 to output the sensitivity information Dn+1.

What is to be selected as candidate information An+1 or how much the information An is to be changed to produce the information An+1 may be determined by using a well-known algorithm such as a genetic algorithm or a bootstrap method.

In the above description, the new information An+1 is produced by changing the information An, but at Step 507, a condition in the information C in addition to the information A may be changed to execute steps S503 to S505 again. For example, more appropriate sensitivity information D may be obtained by changing, as a condition included in the information C, the filler kind or contained amount, the void space ratio, the film volume, or the like to adjust the hardness, flexibility, or volume change easiness of the film.

3.2.6. Case in which Sensitivity Information D Satisfies Predetermined Condition (Output Processing)

When the sensitivity information D satisfies the predetermined condition, the estimation unit 156 of the device 100 may output the information A corresponding to the sensitivity information D at Step S508. In this case, the estimation unit 156 may output, in addition to the information A, information related to the film constitution of the receptor, which is preferable for satisfying certain sensitivity information D, such as the filler ratio, the void space ratio, or the film constitution such as the volume of the receptor.

Then, at Step S509, the estimation unit 156 of the device 100 may output the information E related to a specific molecule structure of the virtual polymer based on SP1 of the output information A.

Specifically, the estimation unit 156 may optionally design one or a plurality of molecule structures of the monomer unit by using a list of correspondence between a functional group and the SP value thereof and the group contribution method and may use one or a combination of the designed monomer units to satisfy the SP value specified at Step S508, thereby producing the information E related to a specific molecule structure of the virtual polymer.

The estimation unit 156 may produce the information E related to a specific molecule structure of the virtual polymer or the virtual monomer unit by using a result of estimation performed in the past; or may produce the information E related to a specific molecule structure of the virtual polymer or the virtual monomer unit by using the list of correspondence between a known monomer unit and the SP value of the monomer unit and the group contribution method and using one or a combination of the monomer units in the list to satisfy the SP value specified at Step S508. More specific examples are described below.

3.2.6.1. Example 1 of Processing of Estimating Molecule Structure of Virtual Polymer or Virtual Monomer Unit

For example, in FIG. 5D, the estimation unit 156 estimates the molecule structure of the virtual polymer or the virtual monomer unit by using a result of estimation performed in the past. In this method, at Step S5091, the estimation unit 156 specifies a similar molecule having an SP value close to the SP value specified at Step S508 from the information E related to a virtual-polymer or virtual-monomer-unit molecule structure estimated for another analyte. The method of similar molecule specification is not particularly limited but examples thereof include a method of using the SP value as a reference and specifying a molecule having an SP value close to the SP value specified at Step S508 by using the x parameter, the Ra parameter, or the like.

Subsequently at Step S5092, the estimation unit 156 sets, as the information E, information related to a polymer molecule structure estimated for the specified similar molecule. Specifically, the information E is specified based on provisional estimation that a molecule structure having a close SP value among receptor molecule structures estimated in the past is suitable as the molecule structure of the current receptor.

Accordingly, it is possible to simply and appropriately produce the information E by using a result of estimation performed in the past. Moreover, when the molecule structure of the monomer unit are optionally designed from scratch, a molecule structure with non-realistic stiffness is obtained, but such a synthesis problem can be solved by using a result of estimation performed in the past.

3.2.6.2. Example 2 of Processing of Estimating Molecule Structure of Virtual Polymer or Virtual Monomer Unit

As another example, in FIG. 5E, for example, the estimation unit 156 produces the information A by using the list of correspondence between a known monomer unit and the SP value of the monomer unit and the group contribution method. In this method, at Step S5093, the estimation unit 156 specifies, from the list of correspondence between a known monomer unit and the SP value of the monomer unit, a monomer unit having an SP value close to the SP value specified at Step S508 when regarded as a single monomer unit.

Subsequently at Step S5094, the estimation unit 156 selects another monomer unit to be copolymerized so that the SP value of the specified monomer unit approaches the SP value specified at Step S508, and produces the composition of the combination of the monomer units as the information E. More specifically, when the SP value specified at Step S508 higher than the SP value of the specified monomer unit, a copolymerized monomer unit having an SP value higher than the SP value of the specified monomer unit is specified and the composition of the combination with which the SP value of the analyte is further approached is produced as the information E. When the SP value specified at Step S508 is lower than SP of the specified monomer unit, a copolymerized monomer unit having an SP value lower than the SP value of the specified monomer unit is specified and the composition of the combination so that SP of the analyte is further approached is produced as E.

Accordingly, without past information, it is possible to simply and appropriately produce the information E by using the list of correspondence between a known monomer unit and the SP value of the monomer unit. One monomer unit having the closest SP value or one or a plurality of monomer units for which the x parameter or the Ra parameter satisfies a certain relation may be selected as the monomer unit specified at Step S5093.

3.1.7. Use of Information E

At Step S310, the estimation unit 156 of the device 100 may output the information E to the input-output interface 130. More specifically, the estimation unit 156 of the device 100 may output and control the information E on a display or may output the information E to another device. The information E may be used, for example, by the same method as exemplarily described in the molecular-skeleton-first method.

4. Program

A program of the present embodiment causes a device to execute the sensitivity estimation step of inputting the candidate information A, the information B, and the reference information C to the model to output the sensitivity information D of the receptor constituted by using the polymer for the analyte, the candidate information A being related to the polymer constituting the receptor, the information B being related to the analyte, the reference information C being related to a film constitution of the receptor.

As described above, in the sensitivity estimation step, the program of the present embodiment may cause the device to additionally input the information F related to mutual interaction between the polymer and the analyte to the model to output the sensitivity information D of the receptor constituted by using the polymer for the analyte.

The program of the present embodiment may be stored and provided in a computer-readable storage medium. The storage medium can store the program in a “non-temporary physical medium”. The program includes a software program and a computer program as non-limiting examples.

The present disclosure is not limited to the above-described embodiment but may be modified in various manners without departing from the scope of the present disclosure. In other words, the above-described embodiment is merely exemplary in any aspects and not to be interpreted in a restrictive manner. For example, the above-described processing steps may be executed in an optionally changed order or in parallel without inconsistency in processing contents.

### EXAMPLES

### Example

A machine learning model was acquired by a random forest model by: having, as explanatory variable, information related to the film constitution of the receptor, such as: the solubility parameter H1 (δt, δd, δp, and θh) of the polymer constituting the receptor; the χ parameter and the Ra parameter; the molecule volume and vapor pressure of the analyte; the coating volume of the receptor; the composition amount of silica; and the particle diameter of silica; preparing, as an objective variable, 1000 kinds of teacher data having highest sensitivity to various single gasses at 250 ppm; and setting 80% of the teacher data as training data and 20% thereof as test data.

Test data not used in the model establishment was input to the acquired model, and sensitivity information was acquired as an output value. Then, a correlation coefficient r² between the actual value of sensor sensitivity checked by actually synthesizing a compound and sensitivity information predicted by the model was checked. As a result, the correlation coefficient r² was calculated to be 0.83 and it was checked that the model in the example has high versatility. FIG. 6A illustrates this result.

Then, a model was established again by using all teacher data, a receptor structure having high sensitivity in 250 ppm of acetone was predicted by using the model, and highest 10 kinds of proposed structures were acquired. When one of the structures was actually synthesized to produce a sensor, the actual value of sensitivity in 250 ppm of acetone was acquired and the produced sensor had sensitivity at least three times higher than that of a receptor (teacher data example) of a molecular skeleton used as typical teacher data.

### Comparative Example

Fitting of fitting parameters was performed with 15 pieces of data randomly extracted by using an expression disclosed in Non Patent Literature, and the rest of the data was used as validation data. With r2=0.64 for the data used for the fitting, r2=−0.46 was obtained, which indicates no versatility for new data (validation data), and the use as a molecular skeleton prediction model was impossible. FIG. 6B illustrates this result.

1. A method that executes, by a device, a sensitivity estimation step of inputting candidate information A, information B, and reference information C to a model to output sensitivity information D of a receptor constituted by using a polymer for an analyte, the candidate information A being related to the polymer, the information B being related to the analyte, the reference information C being related to a film constitution of the receptor.

2. The method according to claim 1, wherein the information B comprises a one-dimensional solubility parameter H2-1 of the analyte.

3. The method according to claim 1, wherein the information B comprises a three-dimensional solubility parameter H2-2 of a London dispersion force term, a dipole moment term, and a hydrogen bond term of the analyte.

4. The method according to claim 1, wherein the information B comprises a Hansen solubility parameter H2-3 of the analyte.

5. The method according to claim 2, wherein
in the sensitivity estimation step, information F related to mutual interaction between the polymer and the analyte is additionally input to the model, and
the information F comprises a x parameter.

6. The method according to claim 4, wherein
in the sensitivity estimation step, information F related to mutual interaction between the polymer and the analyte is additionally input to the model, and
the information F comprises a x parameter and/or a Ra parameter.

7. The method according to claim 1,
wherein in the sensitivity estimation step, the model additionally outputs recommendation information C′ related to the film constitution of the receptor.

8. The method according to claim 1, further executing, before the sensitivity estimation step:
a step of producing a candidate for a molecule structure of the polymer; and
a step of calculating a solubility parameter H1 of the polymer based on the molecule structure produced,
wherein the candidate information A comprises the solubility parameter H1 calculated.

9. The method according to claim 8, wherein the molecule structure comprises a functional group included in the polymer and/or a copolymerization ratio of the polymer.

10. The method according to claim 9, further executing:
a step of outputting the sensitivity information D for each of a plurality of pieces of the candidate information A;
a step of specifying the sensitivity information D that satisfies a predetermined condition among the plurality of pieces of output sensitivity information D; and
a molecule structure estimation step of outputting, as information E related to the molecule structure of the polymer constituting the receptor, the molecule structure of the polymer of the candidate information A corresponding to the specified sensitivity information D.

11. The method according to claim 1, further executing, before the sensitivity estimation step, a step of producing a candidate for a solubility parameter H1 of the polymer based on the information B without specifying the molecule structure of the polymer,
wherein the candidate information A comprises the solubility parameter H1 produced.

12. The method according to claim 11, further executing:
a step of outputting the sensitivity information D for each of a plurality of pieces of the candidate information A;
a step of specifying the sensitivity information D that satisfies a predetermined condition among the plurality of pieces of output sensitivity information D;
a step of estimating the molecule structure of the polymer based on the solubility parameter H1 of the polymer of the candidate information A corresponding to the specified sensitivity information D; and
a molecule structure estimation step of outputting the estimated molecule structure of the polymer as information E related to the molecule structure of the polymer constituting the receptor.

13. A device comprising an estimation unit configured to input candidate information A, information B, and reference information C to a model to output sensitivity information D of a receptor constituted by using a polymer for an analyte, the candidate information A being related to the polymer, the information B being related to the analyte, the reference information C being related to a film constitution of the receptor.

14. The device according to claim 13, wherein
the estimation unit additionally inputs, to the model, information F related to mutual interaction between the polymer and the analyte, and
the information F comprises a x parameter and/or a Ra parameter.

15. A program configured to cause a device to execute a sensitivity estimation step of inputting candidate information A, information B, and reference information C to a model to output sensitivity information D of a receptor constituted by using a polymer for an analyte, the candidate information A being related to the polymer, the information B being related to the analyte, the reference information C being related to a film constitution of the receptor.

16. The program according to claim 15, wherein
in the sensitivity estimation step, information F related to mutual interaction between the polymer and the analyte is additionally input to the model, and
the information F comprises a x parameter and/or a Ra parameter.
